(19) United States
(12) Patent Application Publication
(10) Pub. No.: US 2005/0273151 A1
(43) Pub. Date: Dec. 8, 2005

(54) Stent delivery system

(76) Inventors: John Fulkerson, Rancho Santa Margarita, CA (US); Michael Randall, Mission Viejo, CA (US); Teodoro S. Jimenez JR., Irvine, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/144,513

(22) Filed: Jun. 3, 2005

Related U.S. Application Data
(60) Provisional application No. 60/577,300, filed on Jun. 4, 2004.

(51) Int. Cl. A61F 2/06

(52) U.S. Cl. 623/1.11

(57) ABSTRACT

A stent delivery system includes an inner tubular member on which a stent is loaded, an outer jacket extending over said inner tubular member, the retraction of which causes deployment of the stent, and a handle adapted to move the jacket relative to the inner tubular member. The constructions of the inner tubular member and outer jacket and the handle provide increased control of the relative movement of the outer jacket relative to the inner tubular member, and prevention of premature release of the stent from the deployment instrument, and greater control over stent deployment among other advantages.

### CROSS-REFERENCE TO RELATED APPLICATIONS

The present application claims priority to U.S. Provisional Application No. 60/577,300, filed on Jun. 4, 2004, the disclosure of which is incorporated herein by reference in its entirety for all purposes.

### FIELD OF INVENTION

This invention relates broadly to medical devices. More particularly, this invention relates to an instrument for delivering a self-expanding stent into a mammalian body and controllably releasing the stent.

### BACKGROUND OF THE INVENTION

Transluminal prostheses are widely used in the medical arts for implantation in blood vessels, biliary ducts, or other similar organs of the living body. These prostheses are commonly known as stents and are used to maintain, open, or dilate tubular anatomical structures.

The underlying structure of the stent can be virtually any stent design. There are typically two types of stents: self-expanding stents and balloon expandable stents. Stents are typically formed from malleable metals, such as 300 series stainless steel, or from resilient metals, such as super-elastic and shape memory alloys, e.g., Nitinol™ alloys, spring stainless steels, and the like. They can also, however, be formed from non-metal materials such as non-degradable or biodegradable polymers or from bioresorbable materials such as levorotatory polylactic acid (L-PLA), polyglycolic acid (PGA) or other materials such as those described in U.S. Pat. No. 6,660,827.

Self-expanding stents are delivered through the body lumen on a catheter to the treatment site where the stent is released from the catheter, allowing the stent to automatically expand and come into direct contact with the luminal wall of the vessel. Examples of self-expanding stent suitable for purposes of this invention are disclosed in U.S. Publication No. 2002/0116044, which is incorporated herein by reference. For example, the self-expanding stent described in U.S. Publication No. 2002/0116044 comprises a lattice having two different types of helices (labeled 1-33 in FIG. 1) forming a hollow tube having no free ends. The first type of helix is formed from a plurality of undulations, and the second type of helix is formed from a plurality of connection elements in series with the undulations, wherein the connection elements connect fewer than all of the undulations in adjacent turns of the first type of helix. The first and second types of helices proceed circumferentially in opposite directions along the longitudinal axis of the hollow tube. This design provides a stent having a high degree of flexibility as well as radial strength. It will be apparent to those skilled in the art that other self-expanding stent designs (such as resilient metal stent designs) could be used according to this invention.

The stent may also be a balloon expandable stent which is expanded using an inflatable balloon catheter. Balloon expandable stents may be implanted by mounting the stent in an unexpanded or crimped state on a balloon segment of a catheter. The catheter, after having the crimped stent placed thereon, is inserted through a puncture in a vessel wall and moved through the vessel until it is positioned in the portion of the vessel that is in need of repair. The stent is then expanded by inflating the balloon catheter against the inside wall of the vessel. Specifically, the stent is plastically deformed by inflating the balloon so that the diameter of the stent is increased and remains at an increased state, as described in U.S. Pat. No. 6,500,248 B1, which is incorporated herein by reference.

Stents are delivered to an implant site with the use of a delivery system. Delivery systems for self-expanding stents generally comprise an inner tubular member on which the stent is loaded and which may be fed over a guidewire, and an outer tubular member or jacket longitudinally slidable over the inner tubular member and adapted to extend over the stent during delivery to the implant site. The jacket is retracted along the inner tubular member to release the self-expanding stent from the inner tubular member.

In several available delivery systems, the jacket and inner member are freely movable relative to each other and must be separately manually held in the hands of the physician. After the distal end of the system is located at the implant site, the inner member must be held still to prevent dislocation. However, it is very difficult to maintain the position of the inner member while moving the outer member to deploy the stent. As such, the degree of control during deployment is limited. Under such limited control there is a tendency for the stent to escape from the inner member before the jacket is fully retracted and jump from the desired deployment site. This may result in deployment of the stent at a location other than the desired implant site.

A handle may be provided to move the outer tubular member relative to the inner tubular member with greater control. For example, Medtronic Inc., utilizes a handle which can lock the inner tube and outer jacket relative to each other and effect relative movement of the two to cause deployment of the stent. However, such handles have several shortcomings. First, the handle is not particularly well suited to short stents as there is little fine control. Second, the handle is not well-suited to long stents, e.g., up to 90 mm in length, as the linear control requires the operator to change his or her grip during deployment in order to generate the large relative motion of the tubular components. Third, it is possible for the stent to automatically release before the jacket is fully retracted from over the stent. This is because the super-elastic expansion of the stent causes the stent to slip distally out of the deployment system before the operator retracts the sheath. The result can be an unintentionally rapid and possibly uneven deployment of the stent. Fourth, without reference to a fluoroscope monitoring the stent, there is no manner to determine from the proximal end of the instrument the progress of stent deployment. Fifth, the construction of the inner tubular member and outer jacket may cause the inner member and jacket to be crushed during use. Furthermore, the inner tubular member is subject to compressive force during deployment and may deform while moving the stent from the desired deployment location.

Another stent delivery system can be seen in the commonly owned U.S. patent application Ser. No. 10/189993 Stent Delivery System, filed Jul. 5, 2002, the contents of which are hereby incorporated by reference.

### OBJECTS AND SUMMARY OF THE INVENTION

It is therefore an object of the invention to provide a stent delivery system that permits a high degree of control during deployment of the stent.

It is another object of the invention to provide a stent delivery system which can be operated with a single hand.

It is a further object of the invention to provide a stent delivery system which has inner and outer tubular members which are not subject to undesirable deformation during deployment.

It is also an object of the invention to provide a stent delivery system which has a distal stent mounting portion having high torqueability and high column strength.

It is an additional object of the invention to provide a stent delivery system which is adapted for use with stents of various lengths.

It is a yet another object of the invention to provide a stent delivery system which indicates at the proximal end of the system the progress of stent deployment.

It is yet a further object of the invention to provide a stent delivery system which indicates under fluoroscopy the progress of stent deployment.

In accord with these objects, which will be discussed in detail below, a stent delivery system includes an inner tubular member, an outer jacket over the inner tubular member, and a handle adapted to effect relative longitudinal movement of the jacket and the inner tubular member. The handle includes a stationary member and a longitudinally movable member. The inner tubular member is fixedly coupled to the stationary member, and the jacket is coupled to the movable member. A strain relief sleeve is coupled to the distal end of the stationary member and extends over the jacket.

In accord with preferred aspects of the invention, the stationary member is preferably elongate and adapted to ergonomically fit in either a physician's left or right hand. The movable member is fixed to a belt extending about two sprockets, and one of the sprockets is coupled preferably via one or more gears to knobs located on both sides of the handle. The knobs are situated such that when the handle is held in a hand, one of the knobs may be rotated by the thumb of the same hand of the physician holding the handle to effect single-handed longitudinal movement of the outer jacket relative to the inner tubular member. The gears used in the handle can be chosen to effect more or less longitudinal travel of the outer jacket relative to a given rotational movement of the knobs. That is, the handle can be adapted to conveniently deploy stents of various lengths with a common rotational movement of the knob relative to the handle. The handle also includes a mechanism which produces an audible click as the knob is rotated to provide audible feedback to the physician regarding movement of the outer jacket.

In accord with another preferred aspect of the invention, the proximal portion of the outer jacket is provided with incremental visual indicia. The visual indicia preferably correspond to the length of the stent being deployed. As such, as the jacket is retracted from the inner tubular member and into the handle, the indicia can be seen to move relative to the strain relief. The jacket can also be provided with relief to provide tactile feedback to the physician.

In accord with other preferred aspects of the invention, the inner tubular member and outer jacket are each preferably substantially trilayer constructions. Each preferably includes an inner layer, a middle layer including a flat wire braid, and an outer layer. The trilayer construction provides a combination of flexibility and columnar strength. The inner tubular member includes a reduced diameter portion on which the stent is loaded. A shoulder is defined at the transition of the inner tubular member into its reduced diameter portion, and the shoulder functions as a stop for the stent. The reduced diameter portion also preferably includes a protruding formation adjacent the shoulder. The formation operates to clamp a proximal end of the stent between the inner tubular member and the outer jacket and thereby secure the stent on the inner tubular member until the outer jacket is fully retracted from over the stent.

As such, the stent deployment device provides greater control over stent deployment via visual and auditory feedback at the proximal end of the instrument, increased control of the relative movement of the outer jacket relative to the inner tubular member, and prevention of premature release of the stent from the deployment device.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view of the stent delivery system according to the invention;

FIG. 2 is a side elevation view of the stent delivery system according to the invention;

FIG. 3 is a schematic cross-section view of the distal end of the stent delivery system according to the invention;

FIG. 4 is a side elevation view of a proximal handle portion of the stent delivery system according to the present invention;

FIG. 5 is a disassembled top perspective view of a proximal handle portion of the stent delivery system according to the present invention;

FIG. 6 is a schematic top view of a proximal portion of the outer jacket and the strain relief sleeve of the stent delivery system;

FIG. 7 is a perspective view of a cradle for supporting a handle of the stent delivery system;

FIG. 8 is a perspective view of the cradle of FIG. 7 shown supporting the handle of the stent delivery system;

FIG. 9 is a side perspective view of a stent delivery system according to the present invention;

FIG. 10 is a side perspective view of the stent delivery system of FIG. 9; and

FIG. 11 is a magnified perspective view of area B in FIG. 9.

### DETAILED DESCRIPTION OF THE INVENTION

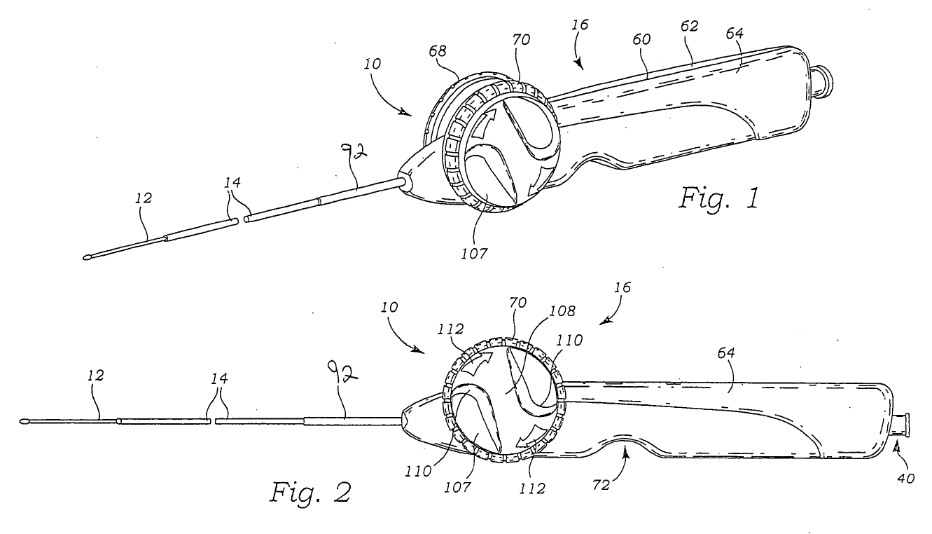

Referring now to FIGS. 1 and 2, a stent delivery system 10 generally includes an inner tubular member 12, a tubular jacket 14 slidable over the inner tubular member 12, and a handle 16 adapted to effect longitudinal movement of the jacket 14 relative to the inner tubular member 12.

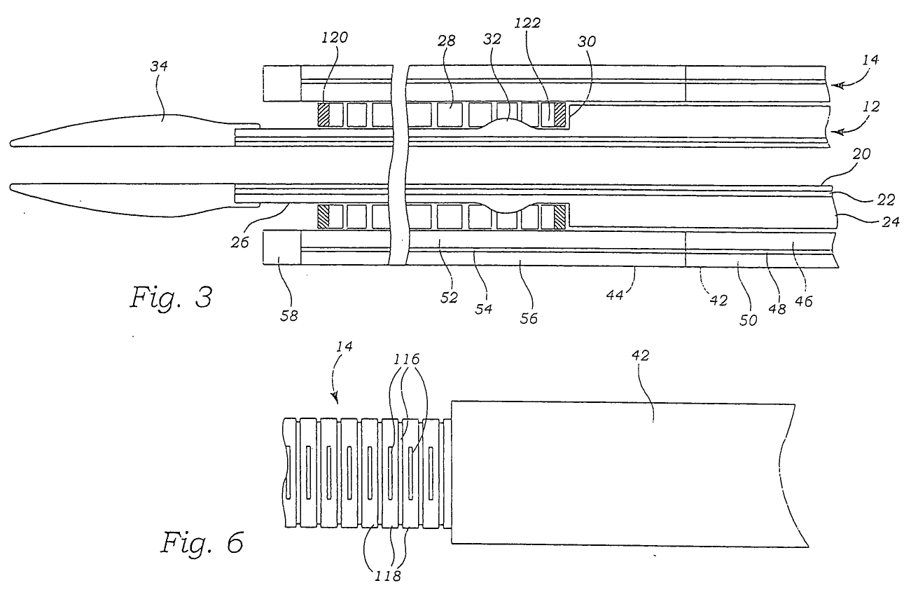

Turning now to FIG. 3, the inner tubular member 12 is preferably a coextruded, trilayer construction. The inner layer 20 is preferably polytetrafluoroethylene (PTFE), fluorinated ethylene propylene (FEP), high density polyethylene (HDPE), or urethane. The middle layer 22 is a wire braid, and more preferably a 304V stainless steel flat wire braid of 1×3 (40 picks) construction, with wires having a 0.001 inch by 0.003 inch rectangular cross-section. Wires of other metals and alloys may also be used, including other stainless steel alloys, cobalt-chrome alloys, and other high-strength, high-stiffness, corrosion-resistant metal alloys. The outer layer 24 is preferably a thermoplastic, melt processible, polyether-based polyamide, such as PEBAX®-7033 available from Modified Polymer Components, Inc. of Sunnyvale, Calif. In the extrusion process, the inner and outer layers are bonded to each other and encapsulate the metallic reinforcing middle wire layer to create an integrated tubing. This tubing exhibits high lateral flexibility combined with a high degree of longitudinal stiffness (resistance to shortening), and also high torqueability. Thus, the inner tubular member is very controllable.

The stent 28 is loaded on a distal portion 26 of the inner tubular member 12 having a reduced diameter created by, for example, centerless grinding, laser grinding, or thermal reduction of the outer layer 24. A shoulder 30 is defined at the transition of the inner tubular member into its reduced diameter distal portion. The shoulder 30 functions as a stop for the stent to prevent the stent from moving proximally on the inner tubular member 12 when the jacket 14 is retracted. The reduced diameter portion also preferably includes a narrow preferably circumferential ridge 32 adjacent the shoulder 30. The proximal end of the stent is frictionally engaged by compression between the ridge of the inner member and the outer sheath. As a result, the stent is prevented from self-advancing out of the delivery system until that ridge of the inner member has been uncovered by the proximally-retracting outer jacket. The distalmost end of the inner tubular member is preferably provided with a tubular soft flexible radiopaque tip 34.

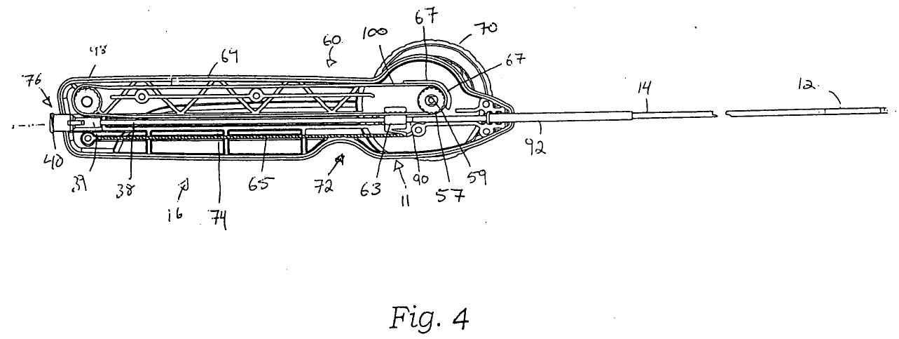

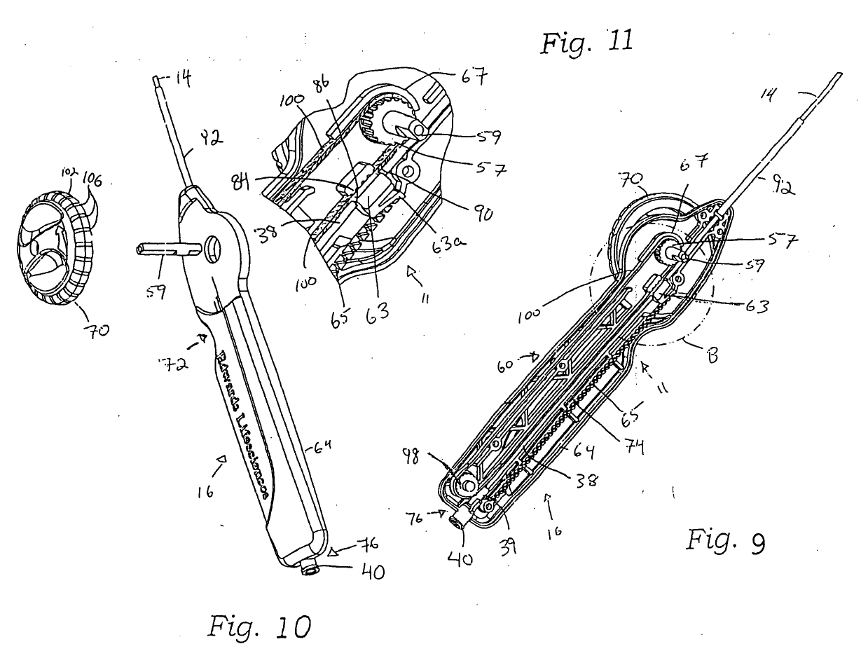

As seen best in FIGS. 4, 9, and 11, a proximal end of the inner tubular member 12 is coupled, e.g., via bonding, to a longitudinally stiff, preferably stainless steel tube 38 of substantially the same outer diameter. The proximal end of the stiff tube 38 is provided with a luer adapter 40 permitting convenient coupling to a mating luer connection and facilitating flushing of the inner tubular member.

Turning back to FIG. 3, the outer jacket 14 includes a first portion 42 extending from its proximal end to near the distal end which preferably has the same trilayer construction as the inner tubular member 12, and preferably a second portion 44 of a different construction adjacent at its distal end. That is, the first portion 42 has an inner layer 46 that is preferably PTFE, FEP, HDPE or urethane, a middle layer 48 that is a preferably stainless steel flat wire braid construction, and an outer layer 50 that is preferably a thermoplastic, melt processible, polyether-based polyamide. The second portion 44 of the outer jacket 14 is preferably a trilayer coextrusion having an inner layer 52 preferably of PTFE, FEP, HDPE or urethane, a middle tie-layer polymer resin 54, such as PLEXAR® available from Equistar Chemicals, LP of Clinton, Iowa, and an outer layer 56 of a thermoplastic, melt processible, polyether-based polyamide. The middle tie-layer resin 54 permits the inner and outer layers 52, 56 to be bonded together into a co-extruded or multilayer composition. The second portion 44 of the outer jacket preferably does not include a braided middle layer, and thus has increased flexibility. In addition, the second portion 44 is preferably a clear construction, permitting visible observation of the stent loaded on the distal portion of the inner tubular member. The first and second portions 42, 44 are preferably substantially seamlessly coupled together using bonding, coextrusion, or other means known in the art; i.e., there are no imperfections at the junction thereof which would interfere with smoothly retracting the outer jacket over the inner tubular member. The distal end of the second portion 44 preferably includes a radiopaque marker 58, such that under fluoroscopy the location of distal end of the jacket relative to fluoroscopically-visible elements of the loaded stent can be monitored. The marker 58 is preferably constructed of a radiopaque metallic material so that it may be crimped securely to the outer jacket. Exemplar suitable materials include platinum, platinum-iridium alloy, tantalum, tantalum-tungsten alloy, zirconium alloy, gold, gold alloy, and palladium, all of which are well-known for use as radiopaque markers in catheter devices.

Referring to FIGS. 1, 2, 4, 5, and 9 the handle 16 generally includes an elongate stationary member 60 defined by two shells portions 62, 64, an internal longitudinally movable member 63, and a pair of manually rotatable wheel-like knobs 68, 70 which effect movement of the movable member 63 relative to the stationary member 60, as described in more detail below.

More particularly, the exterior of the stationary member 60 is preferably ergonomically shaped to fit in the palm of either a left or right hand of an operator and includes a lower grip 72 permitting a pointer finger of the hand of the operator to secure the handle in the palm of the hand. The interior of the stationary member 60 includes an axial track 74 defined by the shell portions 62, 64 of the stationary member 60, and a rear opening 76. The movable member 66 has a preferably substantially cruciate cross-sectional shape, with lateral portions residing in the track 74. An upper portion of the movable member 66 defines a toothed slot 84, and an axial throughbore 86 (FIG. 11) is provided through a central portion of the movable member 63.

As best seen in FIGS. 4, 9, and 11, the stiff tubular portion 38 at the proximal end of the inner tubular member 12 extends through, and is slidable within the axial throughbore 86 of the movable member 63, and a portion of the luer connection 76 is coupled in a pocket 39 at the rear end of the stationary member 60 such that the luer connection 76 extends from the rear of the stationary member 60. As such, the inner tubular member 12 is longitudinally fixed relative to the handle 16, and the stiff tubular portion 38 provides very high longitudinal stiffness at the proximal end of the inner tubular member 12. On the other hand, the outer jacket 14 has a proximal end 90 which is fixedly connected at the axial throughbore 86 of the movable member 63. Thus, when the movable member 63 moves, the outer jacket 14 moves relative to the stationary member 60 of the handle 16. A strain relief sleeve 92 is fixed to the stationary member 60 and extends distally from the stationary member 60. The outer jacket 14 is therefore likewise movable relative to the strain relief sleeve 92.

In addition, the stationary member 60 is provided with a first sprocket 57 at its distal end, and at its proximal end with a second rotating sprocket 98. The first sprocket 57 is mounted on a shaft 59 that extends through the shell portions 62, 64 and receives the knobs 68, 70. A toothed belt 100 extends around the first and second sprockets 57, 98. A portion of the belt 100 is provided in the toothed slot 84 of the movable member 63 to thereby lock the movable member 63 to the belt 100. As a result, rotation of the sprocket 57 causes movement of the belt, which results in movement of the moveable member 66 and movement of the outer jacket 14 relative to the handle 16 and relative to the inner tubular member 12. Alternately, the first and second sprockets 57, 98 may engage the belt 100 by mechanisms other than the gear and tooth method previously described. For example, the first and second sprockets may have friction pads instead of gear teeth to prevent the sprockets from slipping relative to the belt.

An L-shaped bracket 67 (seen best in FIGS. 4, 9, and 11) extends from the inside wall of stationary member 60, partially curving around the first sprocket 57 to prevent the belt 100 from becoming disengaged with the first sprocket 57. Depending on a desired thickness of the belt 100, the L-shaped bracket 67 may be manufactured to have greater or lesser clearance with the sprocket 57.

The stent delivery system 10 may be adjusted to provide different applications of torque, thus varying the speed the outer jacket 14 may be retracted. This variation may be accomplished by substituting the first sprocket 57 for alternate sprockets of varying diameter (not shown). The ratio of the knob 68, 70 diameter to the sprocket 57 diameter will dictate how far the outer sleeve 14 travels for each turn of the knobs 68, 70. A larger diameter sprocket 57 will move the outer sleeve 14 further than a smaller diameter sprocket 57 for the same arc of angular movement of the knobs 68, 70. Accordingly, by using sprockets 57 of alternative diameters, the device can be tailored to provide the deployment characteristics that are optimal for a particular stent product. For example, in one preferred example involving the deployment of a stent of about 200 mm in length, it has been determined that an optimal sprocket 57 diameter is about ⅛th inch for a knob 68, 70 diameter of about 1.95 inches. In another preferred embodiment involving the deployment of a stent of about 30 mm in length, it has been determined that an optimal sprocket 57 diameter is about ½ inches for a knob 68, 70 diameter of about 1.95 inches.

The deployment mechanics on the outer jacket 14 may also be modified by replacing the belt 100 with an alternate belt (not shown) of varying thickness.

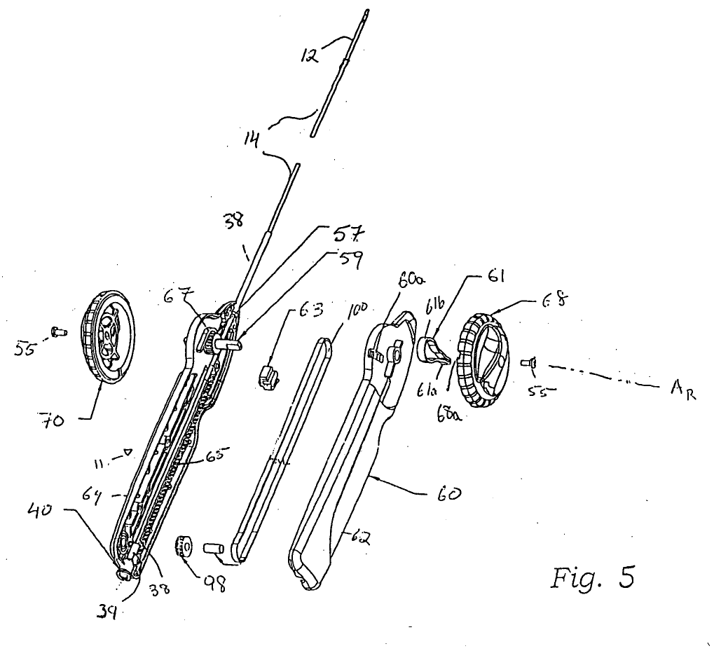

The knobs 68, 70 are provided on each side of the stationary member 60 and connected together with screws 55 (seen best in FIG. 5). Preferably, the knobs 68,70 have a diameter of about 1.95 inches, however other diameters allowing for easy manipulation by a user may alternately be used. The knobs 68, 70 are mounted on an axle 59 and are thus rotatable relative to the stationary member 60, preferably with the axis of rotation A_{R} being vertically offset above the longitudinal axis A_{L} of the stent delivery system 10. Due to the offset of the axis of rotation A_{R} relative to the longitudinal axis A_{L}, the knobs 68, 70 can be kept to a comfortable relatively small size while permitting an upper portion of each knob to rise above the top of the stationary member of the handle. As a result, when the handle 16 is held in either the left or right hand of the physician, the thumb of that hand is situated for placement on one of the knobs. The circumference of the peripheral portion 102 of each knob is preferably entirely exposed (i.e., located outside the stationary member 60) and provided with a friction-enhancing material such as rubber in which is provided a finger engagement structure, such as grooves 106, ribs, or knurls. The respective knob 68, 70 may then be easily rotated by movement of the physician's thumb to effect retraction of the outer jacket 14 relative to the inner tubular member 12. As such, the instrument is adapted for single-handed operation by either hand of the physician.

Nevertheless, it may be desirable by some operators to operate the handle 16 with two hands, one holding the stationary member 60 and the other rotating one of the knobs 68, 70. Therefore, referring to FIG. 2, in order to facilitate this manner of operation, the cover portion 107 of each knob is formed with a raised substantially diametric grip 108 and includes contours 110 adapted to receive a distal portion of thumb to provide leverage in turning the knob. This structure also implicitly identifies the direction of knob rotation for jacket retraction. Moreover, each knob is preferably provided with arrows 112 which explicitly indicate the direction of required rotation.

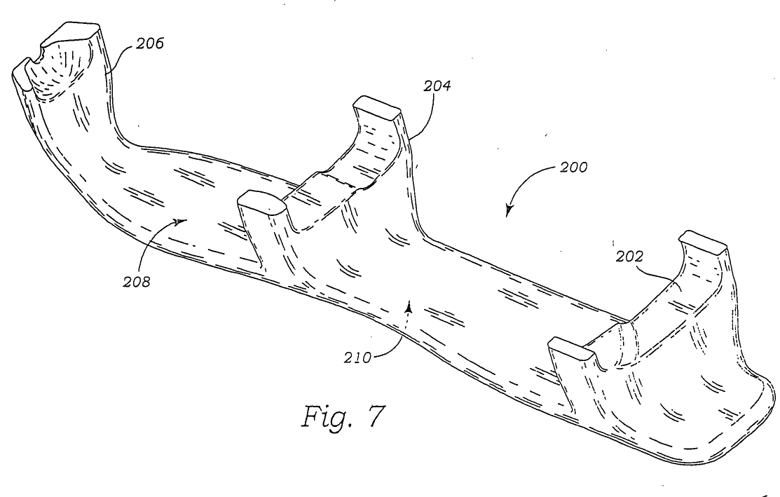

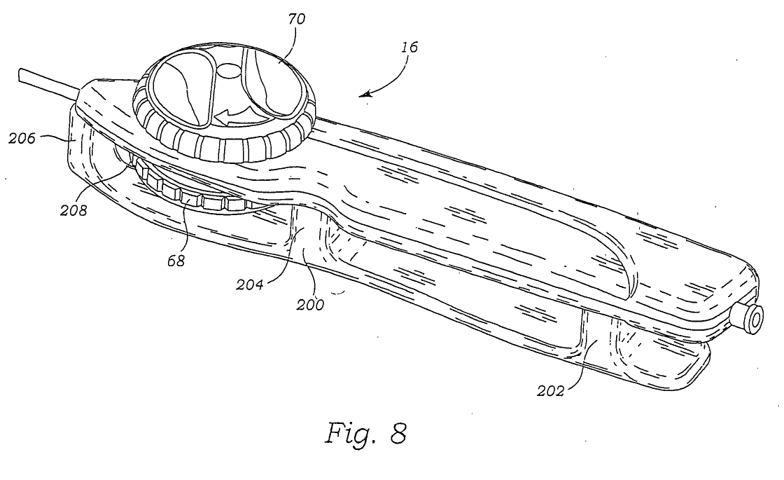

Furthermore, it may be desired by some operators of the instrument to stabilize the handle on a platform, such as the operating table. In accord therewith, referring to FIGS. 7 and 8, a cradle 200 is provided. The cradle 200 includes supports 202, 204, 206 which are adapted to stably hold the handle 16 on its side. When held by the cradle 200, one knob 68 of the handle is received in a space 208, and the other knob 70 faces upward. Knob 68 is positioned in the space 208 such that it freely rotates when knob 70 is manually rotated. The bottom surface 210 of the cradle 200 may be coupled to a platform, e.g., with double-sided adhesive tape. With the handle 16 supported on the cradle 200, the operator may stabilize the handle on the cradle with a hand, and rotate knob 70 to effect stent deployment.

In summary, the handle can be adapted with a gear/pully system wherein the components have different sizes, and different diameters. In this manner, the motion by the operator's hand and corresponding motion of the distal components of the delivery system is adjustable so that the delivery instrument is optimized for each length of stent. Accordingly, the same amount of hand motion by the operator may be translated into relatively less motion in a delivery instrument on which a short stent is loaded, and translated into relatively more motion in a delivery instrument on which a longer stent is loaded. Thus, a common rotational movement may be utilized to deploy stents of any length.

Also according to the invention, the proximal portion of the outer jacket is provided with incremental or quantitative visual indicia 116 (FIG. 6). The visual indicia preferably correspond to the length of the stent being deployed. As such, as the outer jacket 14 is retracted from over the inner tubular member 12 and into the strain relief handle, the indicia can be seen to move relative to the strain relief sleeve 92, and the operator can determine from inspection at the proximal end of the instrument how much of the stent remains to be deployed. The visual indicia may extend only the length of the stent loaded in the system, or may extend the maximum length of any stent which may be loaded on the system, and include discrete markings to indicate the jacket retraction required for deployment of stents of various lengths, e.g., markings at 15 mm, 30 mm, 60 mm, and 90 mm. In addition, the proximal end of the outer jacket may be provided with relief 118, either recessed beneath the surface (as shown) or protruding from the surface, so that the operator may also determine the degree of deployment by tactile feel. The tactile indicia may be coincident or independent of the visual indicia.

Referring now to FIGS. 4, 5, 9, and 11, a one-way slide lock 11 is illustrated according to the present invention. The one-way slide lock 11 allows a user to retract the outer jacket 14, exposing the inner tubular member 12, but locks if the user attempts to move the outer jacket 14 in a distal direction, back over the inner tubular member 12. Thus, during a procedure, a user may uncover a stent 28 by retracting the outer jacket 14 proximally, but may not attempt to recapture the stent 28.

The one-way slide lock 11 comprises a locking movable member 63 that engages locking teeth 65, as best seen in FIG. 11. The locking movable member 63 is coupled to the belt 100 and outer jacket 14 similarly to previously described embodiments of this application. However, as seen best in FIG. 12, the locking movable member 63 includes a locking arm 63a, biased away from the body of the locking movable member 63. As the locking movable member 63 moves proximately, the locking arm 63a contacts a row of locking teeth 65 fixed to the shell portion 64, below the belt 100.

As seen best in FIG. 12, each locking tooth 65 has an angled surface directed distally and a vertical surface on the proximal side. This configuration allows the locking arm 63a to ride over the angled surface, being momentarily urged against the body of locking movable member 63, as the locking movable member 63 travels proximally during a procedure. However, if the belt 100 attempts to move the locking movable member 63 in a distal direction, the locking arm 63a contacts the vertical surface of the locking teeth 65, preventing the biased locking arm 63a from moving back over the locking teeth 65. In this respect, the locking movable member 63 is prevented from distal movement within the stent deployment device, ultimately preventing the outer jacket 14 from moving distally to recapture the stent 28.

According to another aspect of the invention, a locking system is provided to prevent movement of the belt until the system is unlocked. Referring to FIG. 5, a lower side of the stationary member 60 is provided with an opening 60a, and knob 68 includes a notch 68a which when aligned adjacent the opening 60a defines a channel for receiving a spring clip 61. A spring clip 61 includes a resilient U-shaped portion 61a having a barb along one side thereof, and a handle 61b permitting the U-shaped portion 61a to be manually reduced in dimension. When the knob 68 is aligned relative to the opening created by channels 60a and 68a, the U-shaped portion 61a can be placed in the channel with the U-shaped portion 61a being compressed as the barb contacts the area about the opening. The U-shaped portion 61a springs back to shape once seated in the stationary member 60, as the barb seats in a locking notch (not shown). The barb of spring clip 61 interferes with rotation of the knob 68, and thus locks the knobs 68, 70 relative to the stationary member 60. When it is desired to use the device, the clip handle 61b is compressed and the clip 61 is removed.

In use, the distal end of the inner tubular member 12 is fed over a guidewire and guided there along to the deployment site. The distal end of the delivery instrument is then fluoroscopically viewed to ascertain that the instrument is in a predeployment configuration. That is, the delivery instrument is optimized for use with self-expanding stents having a plurality of radiopaque markers 120, 122 at each of its ends, and the ends of the stent are seen to be situated proximal of both the radiopaque tip 34 of the inner tubular member 12 and the radiopaque marker 58 at the distal end of the outer jacket 14 (FIG. 3). One or both of the knobs 68, 70 on the handle 16 is/are then manually rotated relative to the handle to cause retraction of the outer jacket 14. The handle preferably provides audible, tactile, and visual indications of the retraction. Under fluoroscopy, the marker 58 on the jacket 14 is seen to move proximally toward and past the distal stent markers 120. As the stent exits the distal end of the catheter, the distal stent markers 120 are seen to separate radially as the stent 28 self-expands. When the jacket 14 is fully retracted from over the stent 14, the clamping force (created by clamping the proximal end of the stent between the protruding ring 32 on the inner tubular member 12 and the interior of the outer jacket 14) is removed from the proximal end of the stent. When the stent 28 is completely released, the markers 120, 122 at both ends of the stent are seen to be expanded radially, and the marker 58 on the outer jacket is positioned proximal to the markers 122 on the proximal end of the stent.

From the foregoing, it is appreciated that the stent delivery system provides greater control over stent deployment via one or more visual and auditory feedback at the proximal end of the instrument, increased control of the relative movement of the outer jacket relative to the inner tubular member, and prevention of premature release of the stent from the deployment instrument.

There have been described and illustrated herein embodiments of a stent delivery system. While particular embodiments of the invention have been described, it is not intended that the invention be limited thereto, as it is intended that the invention be as broad in scope as the art will allow and that the specification be read likewise. Thus, while particular preferred trilayer constructions for the inner tubular member and outer jacket have been disclosed, it will be appreciated that other constructions, of single or multiple layers and of other materials can be used as well. In addition, while a particular handle configuration has been disclosed, it will be understood that other handles, preferably which permit single-handed operation can also be used. For example, a lower portion of the knobs may be housed within the handle with only a top portion exposed for actuation by an operator's thumb. Furthermore, various aspects of the invention can be used alone without the use of other aspects. For example, the construction of the inner tubular member and outer jacket can be used with delivery systems known in the art, while the preferred handle can be used with conventional inner and outer tubular member constructions. It will therefore be appreciated by those skilled in the art that yet other modifications could be made to the provided invention without deviating from its spirit and scope as claimed.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A stent delivery system, comprising:
a catheter assembly having an inner tubular member and an outer jacket;
said inner tubular member having a distal end sized and shaped to receive a stent;
said outer jacket being longitudinally slidable over said inner tubular member;
a handle body connected to said catheter assembly;
a rotatable member disposed on said handle body and linked through a drive linkage to said outer jacket such that rotation of said rotatable member effects longitudinal movement of said outer jacket relative to said inner tubular member;
said drive linkage including a drive member sized and shaped to effect a predetermined deployment movement of said outer jacket for a predetermined stent.

2. The stent delivery system according to claim 1, wherein said drive member is a replaceable sprocket and wherein each replaceable sprocket has a different diameter.

3. The stent delivery system according to claim 2, wherein said rotatable member has a diameter of about 1.95 inches and wherein said replaceable sprocket has a diameter within the range of about ⅛ inch to about ¼ inch.

4. The stent delivery system according to claim 1, wherein said drive linkage further comprises:
a follower drive member coupled to said drive member by a belt; and,
a movable member disposed on said belt and connected to said outer jacket.

5. The stent delivery system according to claim 1, wherein said drive linkage further comprises a one way lock so as to prevent movement of said outer jacket in a predetermined direction during deployment

6. The stent delivery system according to claim 4, wherein said drive linkage further includes a one way lock so as to prevent movement of said outer jacket in a predetermined direction during deployment and wherein said one way lock includes a biased locking arm extending from said movable member, said biased locking arm being engagable with a plurality of gear teeth disposed in alignment with said movable member on said handle such that said biased locking arm may pass said gear teeth in only one direction.

7. The stent delivery system according to claim 4, wherein a belt guide is disposed in said handle body around said drive member;

8. The stent delivery system according to claim 7, wherein said belt guide and said drive member are spaced from each other by a distance that corresponds substantially to a thickness of said belt.

8. The stent delivery system according to claim 1, wherein said inner tubular member is substantially rigid.

9. A stent delivery system comprising:
a collection of delivery devices having a substantially identical outward appearance;
each of said delivery devices having a drive mechanism for deploying a stent;
at least one of said delivery devices in said collection having a first drive mechanism sized to correspond to a first stent size;
at least one of said delivery devices in said collection having a second drive mechanism sized to correspond to a second stent size; and
said first drive mechanism and said second drive mechanism being sized differently from one another.

10. A stent delivery system according to claim 9, wherein said drive mechanism includes a rotatable drive member.

11. A stent delivery system according to claim 10, wherein said rotatable drive member in said first drive mechanism has a diameter different than the rotatable drive member in said second drive mechanism.

12. A stent delivery system according to claim 11, wherein said rotatable drive member is a sprocket.

13. A stent delivery system according to claim 9, wherein said first stent size is about 20 mm.

14. A stent delivery system according to claim 13, wherein said second stent size is about 200 mm.

15. A stent deployment device comprising:
a catheter assembly having an inner tube and an outer tube, the outer tube being movable relative to said inner tube;
said inner tube having a distal end sized to receive a stent;
a drive mechanism connected to said catheter for moving said outer tube relative to said inner tube and to thereby expose said stent on said inner tube;
said drive mechanism including a one way lock such that said outer tube moves relative to said inner tube in only one direction.

16. A stent deployment device according to claim 15, wherein said drive mechanism further includes a drive member and a follower member coupled together with a belt and a movable member disposed on said belt and fixed to said outer member.

17. A stent deployment device according to claim 16, wherein said one way lock is disposed on said belt.

18. A stent deployment device according to claim 16, wherein said one way lock includes a biasing member located on said movable member and a plurality of teeth disposed on said catheter assembly and matable with said biasing member.

19. A method of deploying a stent comprising:
providing a collection of deployment devices for a plurality of different sized stents; each of said devices in said collection having a substantially identical outward appearance;
selecting at least one deployment device according to a first stent size wherein said deployment device has a drive mechanism tailored for said first stent size, said drive mechanism being different than a drive mechanism for another of said deployment devices;
deploying a stent having said first stent size with said selected deployment device into a patient.

20. A method according to claim 19, further comprising selecting at least one deployment device according to a second stent size, said second stent size being different than a first stent size.
